(19) United States
(12) Patent Application Publication
(10) Pub. No.: US 2012/0277657 A1
(43) Pub. Date: Nov. 1, 2012

(54) BICORPORAL PARTIALLY SUBCUTANEOUS POSITIVE DISPLACEMENT PUMP

(76) Inventors: BRIAN KROETER, PEORIA, IL (US); MICHAEL FLATT, PEORIA, IL (US); DERIC COLE, PEORIA, IL (US)

(21) Appl. No.: 13/460,811

(22) Filed: Apr. 30, 2012

Related U.S. Application Data
(60) Provisional application No. 61/481,043, filed on Apr. 29, 2011.

(51) Int. Cl. A61M 1/00

(52) U.S. Cl. 604/9

(57) ABSTRACT

The present general inventive concept relates to systems and methods for moving a predetermined volume of fluid from one location within a subject's body to another location within a subject's body. The system includes an internal and an external component with no physical connection between. The external component includes the power and control systems while the internal component includes a positive displacement fluid pump that restricts backflow and displaces a controlled volume of fluid over time or per cycle.

### CROSS-REFERENCE TO RELATED APPLICATIONS

This application claims priority pursuant to 35 U.S.C. 119(e) to co-pending U.S. Provisional Patent Application Ser. No. 61/481,043, filed Apr. 29, 2011, the entire disclosure of which is incorporated herein by reference.

### FIELD

The present general inventive concept relates to systems and methods relating to a bicorporal, at least partially subcutaneous, positive displacement pump. More particularly, the present general inventive concept relates to mechanically displacing a controlled volume of body fluid from one location within a subject's body to another location within the subject's body, with a two part pumping system with one part of the pumping system being located external to a subject's body and the other part being located internally within the subject's body.

### BACKGROUND

There are a variety of conditions which result in pathologic chronic collection of bodily fluids within the body of a person. Chronic pericardial effusions, normal pressure hydrocephalus, hydrocephalus, chronic pulmonary effusion, pulmonary edema, and ascites are some of the conditions in which chronic fluid collections persist and result in increased morbidity and mortality.

One common treatment is to drain the bodily fluid to another part of the body, such as the abdomen, stomach, bladder or heart. For example, ventriculoperitoneal (VP) shunts transfer cerebrospinal fluid (CSF) from the ventricles in the brain to the abdomen; lumboperitoneal (LP) shunts transfer CSF from the spinal column into the abdomen; and ventriculoatrial (VA) shunts transfer CSF from the ventricles in the brain to the heart.

Most of the shunts in use today rely on a simple one-way pressure activated valve that is located along a small flexible tube, or catheter to facilitate fluid transfer from areas of higher pressure to areas of lower pressure. The shunts usually include a proximal catheter extending from the check valve to the location of bodily fluid buildup at one end and a discharge catheter extending from the check valve to another part of the body where drainage is acceptable. As bodily fluid collects in one part of the body, pressure builds. If the pressure is at or above a certain threshold level, the pressure sensitive check valve opens, allowing fluid to transfer to the acceptable drainage location.

There are many problems with the currently available shunts, leaving much room for modifications and improvements. Some of the most common problems encountered include mechanical failures, obstruction, infection, over-drainage and frequent corrective surgeries. Due to the nature of the implant any sort of failure will result in eventual symptom recurrence and results from either over or under drainage.

Blockage is most common in the proximal catheter, but any part of the shunt may become blocked. If a shunt becomes obstructed, the CSF is unable to flow through the shunt and it cannot be drained properly. A blocked shunt causes the original symptoms to return, and it may cause additional symptoms such as headaches, nausea, vomiting, and drowsiness.

Infection is always a risk, even though precautions are taken during the surgery to make the shunt sterile, because using a shunt requires insertion of a foreign object into the body. Symptoms of shunt infection are swelling and redness at the incision wounds or along the length of the shunt. In some cases, shunt infection can be treated with intense antibiotic therapy, but in most cases, the shunt needs to be removed.

Over-drainage can occur when a patient is standing, rather than in a lying position and/or when a patient moves from a supine position to an upright one. This change introduces an additional pressure difference between the ventricles and the drainage point, frequently resulting in a valve opening at a lower pressure than desirable and over-drainage of CSF in the patient. Symptoms of over-drainage include vomiting, drowsiness, changes in vision, and a headache that worsens when a patient stands up compared to lying down.

One problem with the current shunt treatment occurs when a patient fails to respond to the shunt implant. The patient's failure to respond to the treatment may be due to the shunt malfunctioning, being clogged, or otherwise allowing little or no fluid to flow through it. Alternatively, the pressure setting of the shunt may be incorrect, requiring adjustment in order to drain more fluid. Then there is always the possibility that the patient simply will not respond to or benefit from a shunt as a method of treatment. Without additional surgery, it is difficult to determine which of these reasons may be causing the shunt treatment to fail.

One example of such a pressure-dependent pump is U.S. Pat. No. 7,621,886 to Burnett. The Burnett patent relies on fluid pressure to trigger a check valve to engage the pump. As noted previously, this can fail when the pressure changes due to environment or patient positioning. It is also difficult to impossible to measure actual volume of fluid displacement with a pressure-dependent pump.

What is needed is a shunt system and method where the volume of fluid being transferred is controllable and measurable without further bodily intrusion and without reliance on fluid pressure.

### SUMMARY

Objects of the present inventive concept include, but are not necessarily limited to, systems and methods related to a bicorporal, at least partially subcutaneous, positive displacement pump. The present general inventive concept relates to mechanically displacing a controlled, predetermined volume of body fluid from one location within a subject's body to another location within the subject's body, with a two part pumping system with one part of the pumping system being located external to a subject's body and the other part being located internally within the subject's body.

In one aspect, a system for moving bodily fluid consistently and reliably without the use of an subcutaneous power supply is provided. The system includes two separate components. One is subcutaneous. Preferably, the subcutaneous, internal component is placed within a subject's body near the outer wall of the abdomen. The other is external.

The internal component includes an inlet and an outlet connected to a fluid chamber. The chamber includes a positive displacement pump that is driven by the external component. Preferably, the drive mechanism is a magnetic induction motor. The pump is configured to displace a predetermined, fixed amount (volume) of fluid over a period of time or per each cycle. Examples of such pump include a peristaltic pump (or roller pump), a piston pump, and a diaphragm pump. The external component includes a power supply and control system to provide the driving force, wirelessly, to drive the pump.

A user controls the internal pump with the external component. The user positions the external component close to the internal component such that the driving force will drive the internal pump. The user powers pump for either a predetermined period of time or a predetermined number of cycles. The user determines the volume of fluid flow through the first pump based on the time or the number of cycles.

Fluid flows only when the system is powered. When the system is not powered, no fluid flows through the system. The pump is configured to protect against backflow. Optionally, the control system also includes a backflow control option to reverse the direction of fluid flow in order to combat inlet catheter obstructions.

The internal component is self-contained and has no physical contact with the second component, nor does it have any power source contained within the subject's body. The control system is easy to use, preferably a single input variable (i.e. running time or number of cycles) based off of doctor prescription provided by the user. The user controls the volume of fluid flow based on either a prescription from a medical professional or feedback from the subject, which may be either objective or subjective.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention and various features thereof.

### BRIEF DESCRIPTION OF THE DRAWINGS

These and/or other aspects and utilities of the present general inventive concept will become apparent and more readily appreciated from the following description of the embodiments, taken in conjunction with the accompanying drawings. For the purpose of illustration, forms of the present general inventive concept which are presently preferred are shown in the drawings; it being understood, however, that the general inventive concept is not limited to the precise arrangements and instrumentalities shown. In the drawings:

FIG. 1 is an exemplary embodiment of the present general inventive concept with a peristaltic pump.

FIG. 2 is another exemplary embodiment of the present general inventive concept with a peristaltic pump.

FIG. 3 is another exemplary embodiment of the present general inventive concept with a piston pump.

FIG. 4 is another exemplary embodiment of the present general inventive concept with a diaphragm pump.

FIG. 5 is a photograph of a laboratory experimental setup to test an embodiment of the present general inventive concept.

FIG. 6 is a diagram of a laboratory experimental setup to test an embodiment of the present general inventive concept.

FIG. 7 is a photograph of an exemplary placement of an embodiment of the present general inventive concept within a subject's body.

### DETAILED DESCRIPTION

As required, a detailed embodiment of the present invention is disclosed herein; however, it is to be understood that the disclosed embodiment is merely exemplary of the principles of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

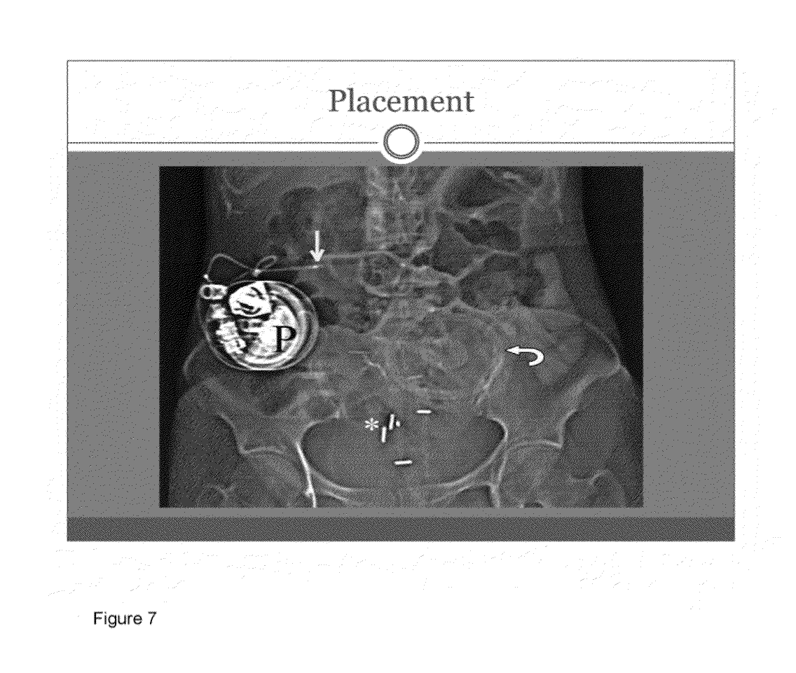

The general inventive concept provides a system for moving bodily fluid from one part of a subject's body to another part of the subject's body consistently and reliably without the use of an subcutaneous power supply. The system includes two separate components. A first component is situated subcutaneously, or in other words, beneath a subject's skin. FIG. 7 shows an example of a preferred placement within a subject's body near the outer wall of the abdomen.

The first component is an internal component and, therefore, is made of biocompatible materials. The first component is sized and shaped such that it can be placed within a body cavity, such as the abdomen. The first (i.e., subcutaneous) component includes an inlet and an outlet operably connected to (i.e., in fluid communication with) a chamber through which fluid is moved from the inlet to the outlet. The inlet is operably connected to (i.e., in fluid communication with) a part of the subject's body, via a catheter, for example. The outlet is also operably connected to a part of the subject's body. For example, and not by way of limitation, a catheter may extend from the subject's brain or spinal cord to the inlet and another catheter may extend from the outlet to the subject's belly or abdomen. In this example fluid can flow from the brain or spinal cord, through the first catheter to the inlet, through the chamber of the first component, through the outlet, through the second catheter and discharge into the subject's belly or abdomen.

The chamber includes a positive displacement pump that is driven by an external force, such as a magnetic induction motor. The positive displacement pump is configured to displace a predetermined fixed amount (volume) of fluid over a period of time or cycle. One example of such a positive displacement pump is a peristaltic pump (or roller pump). Another example of such a positive displacement pump is a piston pump. Another example of such a positive displacement pump is a diaphragm pump.

The second component is situated external to the subject's body. The second component includes a power supply and control system to provide a driving force, wirelessly and external to the subject's body, to drive the positive displacement pump of the first component. One example of the second component providing the wireless driving force is a magnetic induction motor. The example of the magnetic induction motor is capable of providing the driving force through the subject's body, but without breaking the skin. The magnetic induction motor example provides a magnetic field, preferably over a maximum of three inches of a material such as the abdominal wall of the subject's body. The second component is powered directly via AC or DC electrical current. In some embodiments, the second component is incorporated within a belt or case that is strapped to the outside of subject's body.

A user selectively controls the pump by operation of the second (i.e., external) component. The user brings the second component to within sufficient proximity of the first (i.e., internal) component that the driving force will drive the positive displacement pump of the first component. The user powers the first component for either a predetermined period of time or a predetermined number of cycles. The user determines the volume of fluid flow through the first (i.e., internal) component based on the predetermined period of time or the predetermined number of cycles.

The first component is configured such that fluid flow can occur only when the system is powered. When the system is not powered, no fluid flows through the system. They first component is configured to minimize backflow to within acceptable safety standards. A preferred embodiment of the chamber of the first component is approximately equal in size to a cylinder having a three inch diameter and one inch height. Preferably, the daily flow volume is between 100 and 200 mL and occurs at a maximum flow rate of 6.25 mL per minute and a minimum of 3.00 mL per minute. To prevent potentially fatal over-drainage and to prevent the risk of contamination and further complications, no flow occurs when the system is unpowered. Optionally, the control system also includes a backflow option to reverse the direction of fluid flow within the first component in order to combat inlet catheter obstructions.

The first component is self-contained and has no physical contact with the second component, nor does it have any power source contained within the subject's body. Ideally the first component has a working life longer than that of the subject, however, a first component with a working life of less than that of the subject is acceptable under certain conditions. While the specific flow volume required will ultimately be based off of the individual subject, the maximum flow rate does not exceed 6.25 mL/minute and is reasonably accurate over the length of the run (within 10% of the predicted output volume for the operating time). The control system is easy to use, preferably a single input variable (i.e. running time or number of cycles) based off of doctor prescription is provided by the user. The user controls the volume of fluid flow based on either a prescription from a medical professional (for example, 10 minutes every 12 hours) or feedback from the subject. Feedback from the subject is either objective (such as pressure readings) or subjective (such as the subject feels dizzy or tired).

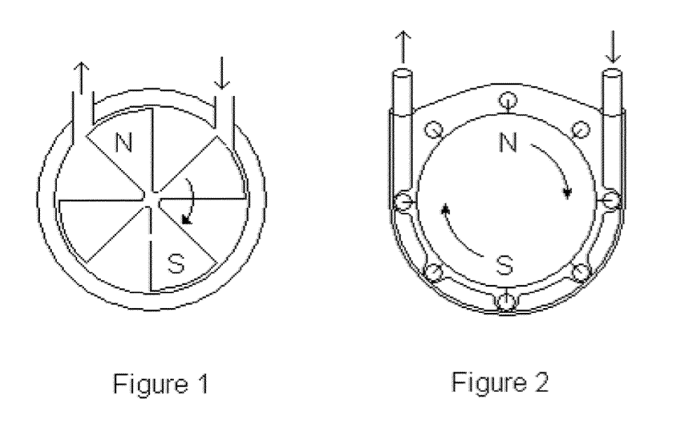

Referring to the drawings, FIG. 1 shows an example of a peristaltic positive displacement pump of an embodiment of the general inventive concept. According to FIG. 1, the pump includes an inlet, an outlet and a chamber through which fluid is pumped from the inlet to the outlet. The pump includes a plurality of rotatable blades or fins. Between each fin is a predetermined volume of space capable of being filled with fluid. As the fins rotate clockwise, the space in fluid communication with the inlet fills with fluid. As the fins continue to rotate clockwise, the fluid in the space between the fins also rotates clockwise until it reaches fluid communication with the outlet. As the fins continue to rotate clockwise, the fluid in the space between the fins is discharged through the outlet. According to FIG. 1, the fins are magnetized and driven by a rotating magnetic field generated by a magnetic induction motor.

FIG. 2 shows another example of a peristaltic positive displacement pump of an embodiment of the general inventive concept. According to FIG. 2, the pump includes flexible tubing extending from the inlet to the outlet. Rather than the fins of FIG. 1, FIG. 2 includes rollers that pinch or restrict the flexible tubing at various locations between the inlet and outlet. According to FIG. 2, as the rollers rotate clockwise, discrete volumes of fluid are pumped from the inlet, through the flexible tubing and discharged through the outlet. Similar to FIG. 1, the pump of FIG. 2 is powered by a rotating magnetic field of a magnetic induction motor. The pump of FIG. 2 obstructs (prevents) fluid flow when the system is unpowered. The pump of FIG. 2 may reverse the direction of fluid flow, even if only temporarily to remove an inlet obstruction, by reversing the direction of the rotating magnetic field driver.

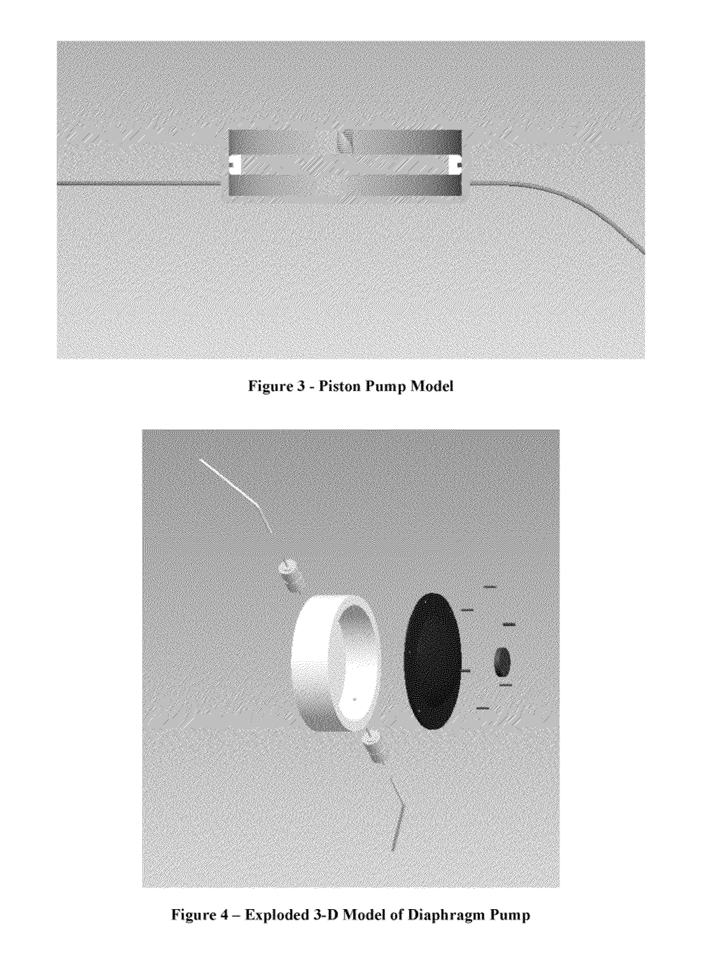

Referring to FIG. 3, an example of an embodiment of the general inventive concept with a piston pump is shown. According to FIG. 3, the pump is a piston and cylinder device where the main piston has a magnetic component attached and is also attached to a spring. The power and controller component located outside the body is an electromagnet powerful enough to displace the piston. According to FIG. 3, the housing, spring, and piston is machined from nonmagnetic materials to eliminate any additional induced magnetic force. Two one-way pressure-check valves are placed in series on the housing serving as an inlet and outlet respectively. A strong disc-shaped rare earth magnet is attached to the piston in order to allow interactive control via the electromagnet. Activating the electromagnet causes the piston to move towards the electromagnet, compressing the spring and creating a vacuum pressure to open the inlet check valve to allow a predetermined volume of fluid flow into the chamber. As the piston reaches the bottom dead center position, the electromagnet deactivates momentarily. As the pump chamber is filled to the predetermined volume, the pressure differential closes the inlet check valve. The spring restoring force returns the piston to its original position, displacing the fluid in the chamber out of the exit valve which opens as fluid pressure builds behind it. The cycle repeats itself as needed upon reapplication of the magnetic field from the electromagnet.

Referring to FIG. 4, an example of an embodiment of the general inventive concept with a diaphragm pump is shown. The diaphragm pump embodiment of FIG. 4 operates on similar principles to the piston pump of FIG. 3 by simply substituting a flexible diaphragm for the piston and spring. The piston and the spring from the piston design of FIG. 3 are functionally replaced in FIG. 4 by the diaphragm material which serves to simplify the design considerably by requiring fewer moving parts. According to FIG. 4, the diaphragm attaches to a cylinder machined out of PVC plastic via threads in order to form a water tight seal, and then a small rare earth magnetic is attached to the top of the diaphragm with an adhesive in order to operate it in conjunction with the electromagnetic controller in a similar process to the piston design of FIG. 3. The cyclical operation process of the diaphragm of FIG. 4 is nearly identical to the piston design of FIG. 3, with two check valves connected in series directly to the housing via adhesive, except the diaphragm material itself, or, if the controller allows it, the electromagnetic force, causes the diaphragm to rebound to its initial position. A multitude of diaphragm options exist; however, a 3.5 inch sanoprene diaphragm is preferred because of the size constraints coupled with its extremely high service life.

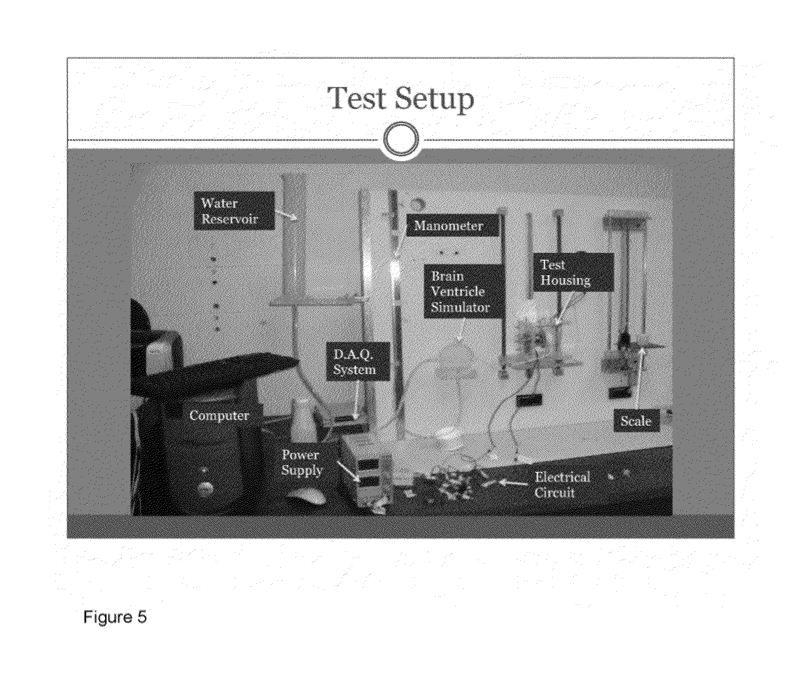

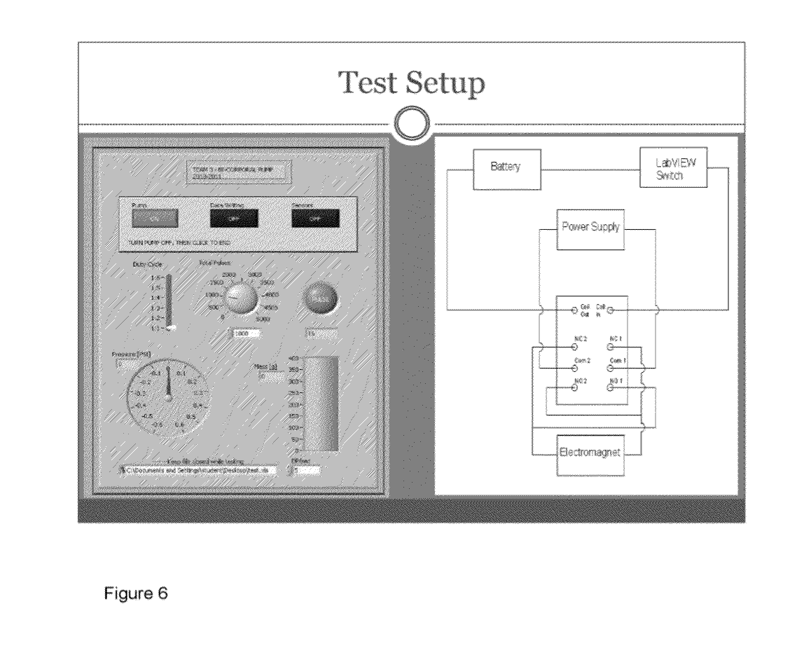

Referring to FIGS. 5 and 6, a test setup provides a simulation of both the path that cerebrospinal fluid travels in a normal human body and the driving pressures present. To accurately represent a variety of in vivo pressures and the effect changes in patient position have on pressure an adjustable shelf with a water reservoir is placed above the pump. A tube runs from the reservoir and feeds into a small volume that simulates the patients' ventricular cavity. The proximal end of the catheter is placed within this container on the opposing side of the reservoir tube. From there the catheter tubing extends to the inlet of the pump and, after passing through the housing, into a drainage container. The drainage container is located on a scale so that the mass and, consequently, the volume of displaced fluid is recorded. The system is controlled by a modified version of the LabVIEW program. With several adjustable inputs and outputs, the program is set up to send voltage pulses to a pump while also recording a differential pressure and the mass of the fluid in the drainage container. The frequency of the pulses is adjustable and is set by the duty cycle. The electromagnet powers the actual pump through the same voltage pulses, displacing the diaphragm and causes the pumping motion. A standard test cycle is set for the pump, and each test results in a similar displacement of fluid upon completion. The number of pulses required for a standard operating cycle is determined based on the average amount of fluid that is displaced with each cycle. Inadequate fluid displacement will result in an overall increase in the number of pulses that will be required for each operating cycle. Successful tests are based upon the accuracy of each run and if the required flow rate was reached.

### Calculations

The following calculations provide a basis for optimization of fluid flow through the system.

### Assumptions

Fluid has the properties of water at STP.

### Flow Rates

Where Qₘₐₓ and Qₘᵢₙ are the minimum and maximum acceptable flow rates listed in [mL/min] and [fl oz/min].

Through Entire System:

$$\begin{matrix}\begin{matrix}{Q}_{ma x} = [ \frac{50 mL}{8 min} ] * \frac{1}{8} \\ = 6.25 [ \frac{mL}{min} ] \\ = 6.25 [ \frac{mL}{min} ] * \frac{1 [ fl oz ]}{29.573 [ mL ]} \\ = 0.211 [ \frac{fl oz}{min} ]\end{matrix} & Eqn . 1 \\ {Q}_{m ax} = 6.25 [ \frac{mL}{min} ] = 0.211 [ \frac{fl oz}{min} ] & Eqn . 2 \\ \begin{matrix}{Q}_{m i n} = [ \frac{25 mL}{8 min} ] * \frac{1}{8} \\ = 3.13 [ \frac{mL}{min} ] \\ = 3.13 [ \frac{mL}{min} ] * \frac{1 [ fl oz ]}{29.573 [ mL ]} \\ = 0.106 [ \frac{fl oz}{min} ]\end{matrix} & Eqn . 3 \\ {Q}_{m i n} = 3.13 [ \frac{mL}{min} ] = 0.106 [ \frac{fl oz}{min} ] & Eqn . 4\end{matrix}$$

### Average Fluid Velocities

$$\begin{matrix}V = \frac{Q}{A} & Eqn . 5 \\ A = \frac{π}{4} * {D}^{2} & Eqn . 6\end{matrix}$$

where:

V is the velocity [m/s]
Q is the volumetric flow rate [m³/s]
A is the cross-sectional area [m²]
D is the diameter [m]

### Through Check Valves:

$$\begin{matrix}D = .125 [ in ] * \frac{.0254 [ m ]}{1 [ in ]} = .00318 [ m ] & Eqn . 7 \\ A = \frac{π}{4} * {( 0.00318 [ m ] )}^{2} = 7.94 * {10}^{- 6} [ {m}^{2} ] & Eqn . 8 \\ \begin{matrix}{V}_{m ax} = \frac{( 6.25 [ \frac{mL}{min} ] * \frac{1 * {10}^{- 6} [ {m}^{3} ]}{1 [ mL ]} * \frac{1 [ min ]}{60 [ s ]} )}{7.94 * {10}^{- 6} [ {m}^{2} ]} \\ = .0131 [ \frac{m}{s} ] \\ = .0131 [ \frac{m}{s} ] * \frac{1 [ ft ]}{.3048 [ m ]} \\ = .0430 [ \frac{ft}{s} ]\end{matrix} & Eqn . 9 \\ {V}_{m ax} = .0131 [ \frac{m}{s} ] = .0430 [ \frac{ft}{s} ] & Eqn . 10 \\ \begin{matrix}{V}_{m i n} = \frac{( 3.13 [ \frac{mL}{min} ] * \frac{1 * {10}^{- 6} [ {m}^{3} ]}{1 [ mL ]} * \frac{1 [ min ]}{60 [ s ]} )}{7.94 * {10}^{- 6} [ {m}^{2} ]} \\ = .00657 [ \frac{m}{s} ] \\ = .00657 [ \frac{m}{s} ] * \frac{1 [ ft ]}{.3048 [ m ]} \\ = .0193 [ \frac{ft}{s} ]\end{matrix} & Eqn . 11 \\ {V}_{m i n} = .00657 [ \frac{m}{s} ] = .0193 [ \frac{ft}{s} ] & Eqn . 12\end{matrix}$$

### Through Catheter Tubing

$$\begin{matrix}D = .0015 [ m ] & Eqn . 13 \\ A = \frac{π}{4} * {( 0.0015 [ m ] )}^{2} = 1.77 * {10}^{- 6} [ {m}^{2} ] & Eqn . 14 \\ \begin{matrix}{V}_{m ax} = \frac{( 6.25 [ \frac{mL}{min} ] * \frac{1 * {10}^{- 6} [ {m}^{3} ]}{1 [ mL ]} * \frac{1 [ min ]}{60 [ s ]} )}{1.77 * {10}^{- 6} [ {m}^{2} ]} \\ = .0589 [ \frac{m}{s} ] \\ = .0589 [ \frac{m}{s} ] * \frac{1 [ ft ]}{.3048 [ m ]} \\ = .193 [ \frac{ft}{s} ]\end{matrix} & Eqn . 15 \\ {V}_{m ax} = .0589 [ \frac{m}{s} ] = .193 [ \frac{ft}{s} ] & Eqn . 16 \\ \begin{matrix}{V}_{m i n} = \frac{( 3.13 [ \frac{mL}{min} ] * \frac{1 * {10}^{- 6} [ {m}^{3} ]}{1 [ mL ]} * \frac{1 [ min ]}{60 [ s ]} )}{1.77 * {10}^{- 6} [ {m}^{2} ]} \\ = .0295 [ \frac{m}{s} ] \\ = .0295 [ \frac{m}{s} ] * \frac{1 [ ft ]}{.3048 [ m ]} \\ = .0967 [ \frac{ft}{s} ]\end{matrix} & Eqn . 17 \\ {V}_{m i n} = .0295 [ \frac{m}{s} ] = .0967 [ \frac{ft}{s} ] & Eqn . 18\end{matrix}$$

Reynolds Number

$$Re = \frac{ρ * V * D}{μ} \qquad Eqn . 19$$

where:

Re is the Reynolds number
ρ is the density [kg/m³]
D is the diameter [m]
μ is the dynamic viscosity [Pa*s]

### Through Check Valves

$$\begin{matrix}\begin{matrix}{Re}_{ma x} = \frac{( 1000 [ \frac{kg}{{m}^{3}} ] * .0131 [ \frac{m}{s} ] * .00318 [ m ] )}{1.002 * {10}^{- 3} [ Pa * s ]} \\ = 41.66 ( Laminar )\end{matrix} & Eqn . 20 \\ \begin{matrix}{Re}_{m i n} = \frac{( 1000 [ \frac{kg}{{m}^{3}} ] * .00657 [ \frac{m}{s} ] * .00318 [ m ] )}{1.002 * {10}^{- 3} [ Pa * s ]} \\ = 20.85 ( Laminar )\end{matrix} & Eqn . 21\end{matrix}$$

### Through Catheter Tubing

$$\begin{matrix}\begin{matrix}{Re}_{ma x} = \frac{( 1000 [ \frac{kg}{{m}^{3}} ] * .0589 [ \frac{m}{s} ] * .0015 [ m ] )}{1.002 * {10}^{- 3} [ Pa * s ]} \\ = 88.17 ( Laminar )\end{matrix} & Eqn . 22 \\ \begin{matrix}{Re}_{m i n} = \frac{( 1000 [ \frac{kg}{{m}^{3}} ] * .0295 [ \frac{m}{s} ] * .0015 [ m ] )}{1.002 * {10}^{- 3} [ Pa * s ]} \\ = 44.16 ( Laminar )\end{matrix} & Eqn . 23\end{matrix}$$

Electromagnetic Contact Force Required (Theoretical)

F_{c}=F_{d}*D³ Eqn. 24

where:

F_{d} is the force at a distance D [lbf]
F_{c} is the contact force of the electromagnet [lbf]
D is the distance [in] set at 3 [in]
Piston (Assuming F_{c}=8 lbf)

$$\begin{matrix}\begin{matrix}{F}_{c} = 8 [ lbf ] * {( 3 [ in ] )}^{3} \\ = 216 [ lbf ] \\ = 216 [ lbf ] * \frac{4.45 [ N ]}{1 [ lbf ]} \\ = 961.2 [ N ]\end{matrix} & Eqn . 25 \\ {F}_{c} = 216 [ lbf ] = 961.2 [ N ] & Eqn . 26\end{matrix}$$

Diaphragm (Assuming F_{c}=5 lbf)

$$\begin{matrix}\begin{matrix}{F}_{c} = 5 [ lbf ] * {( 3 [ in ] )}^{3} \\ = 45 [ lbf ] \\ = 45 [ lbf ] * \frac{4.45 [ N ]}{1 [ lbf ]} \\ = 200 [ N ]\end{matrix} & Eqn . 27 \\ {F}_{c} = 45 [ lbf ] = 200 [ N ] & Eqn . 28\end{matrix}$$

Pressure Drops (Theoretical)

$$\begin{matrix}Δ P = f * ( \frac{L}{D} ) * ( \frac{ρ * {V}^{2}}{2} ) & Eqn . 29 \\ f = \frac{64}{Re} & Eqn . 30\end{matrix}$$

where:

ΔP is the pressure drop [Pa]
f is the friction factor
L is the pipe or tubing length [m]
D is the pipe or tubing diameter [m]
V is the fluid velocity [m/s]

### Through the Entire System

Note: All pressures are gage pressures

$$\begin{matrix}\begin{matrix}{P}_{inlet} = 200 [ cm {H}_{2} O ] \\ = 200 [ cm {H}_{2} O ] * \frac{98.06 [ Pa ]}{1 [ cm {H}_{2} O ]} \\ = 19612 [ Pa ] \\ = 19612 [ Pa ] * \frac{0.000145 [ psi ]}{1 [ Pa ]} \\ = 2.84 [ psi ]\end{matrix} & Eqn . 31 \\ {P}_{inlet} = 19612 [ Pa ] = 2.84 [ psi ] & Eqn . 32 \\ {P}_{outlet} = {P}_{at m} = 0 [ Pa ] = 0 [ psi ] & Eqn . 33 \\ \begin{matrix}Δ {P}_{total} = {P}_{inlet} - {P}_{outlet} \\ = 19612 [ Pa ] - 0 [ Pa ] \\ = 19612 [ Pa ] \\ = 2.84 [ psi ]\end{matrix} & Eqn . 34 \\ Δ {P}_{total} = 19612 [ Pa ] = 2.84 [ psi ] & Eqn . 35\end{matrix}$$

Through Catheter Tubing Up to Pump (at Maximum Velocity)

$$\begin{matrix}f = \frac{64}{88.17} = 0.726 & Eqn . 36 \\ \begin{matrix}Δ {P}_{m ax tubing } = 0.726 * ( \frac{0.4752 [ m ]}{.0015 [ m ]} ) * \\ ( \frac{1000 [ \frac{kg}{{m}^{3}} ] * {( .0589 [ \frac{m}{s} ] )}^{2}}{2} ) \\ = 398.96 [ Pa ] \\ = 398.96 [ Pa ] * \frac{0.000145 [ psi ]}{1 [ Pa ]} \\ = 0.0579 [ psi ]\end{matrix} & Eqn . 37 \\ Δ {P}_{ma x tubing} = 398.96 [ Pa ] = 0.0579 [ psi ] & Eqn . 38\end{matrix}$$

Note this is equal to the pressure drop through the catheter tubing after the pump as well, because the length, diameter, friction factor, and velocity do not change. Therefore:

ΔP_{max total tubing}=398.96 [Pa]*2=797.92 [Pa]=0.116 [psi] Eqn. 39

Through Pump (at Maximum Velocity)

ΔP_{max pump}=ΔPₜₒₜₐₗ−ΔP_{max total tubing}=19612 [Pa]=797.92 [Pa]=18814.1 [Pa]=2.73 [psi] Eqn. 30

Through Catheter Tubing Up to Pump (at Minimum Velocity)

$$\begin{matrix}f = \frac{64}{44.16} = 1.45 & Eqn . 31 \\ \begin{matrix}Δ {P}_{m i n tubing} = 1.45 * ( \frac{0.4752 [ m ]}{.0015 [ m ]} ) * \\ ( \frac{1000 [ \frac{kg}{{m}^{3}} ] * {( .0295 [ \frac{m}{s} ] )}^{2}}{2} ) \\ = 199.88 [ Pa ] \\ = 398.96 [ Pa ] * \frac{0.000145 [ psi ]}{1 [ Pa ]} \\ = 0.0290 [ psi ]\end{matrix} & Eqn . 32 \\ Δ {P}_{m i n tubing} = 199.88 [ Pa ] = 0.0290 [ psi ] & Eqn . 33\end{matrix}$$

Note this is equal to the pressure drop through the catheter tubing after the pump as well, because the length, diameter, friction factor, and velocity do not change. Therefore:

ΔP_{min total tubing}=199.83 [Pa]=2=399.76 [Pa]=0.0580 [psi] Eqn. 34

Through Pump (at Minimum Velocity)

ΔP_{min pump}=ΔPₜₒₜₐₗ−ΔP_{min total tubing}=19612 [Pa]−399.76 [Pa]=19212.2 [Pa]=2.79 [psi] Eqn. 30

Ideal Work Done by Pump (Theoretical)

W=Q*ΔP Eqn. 31

where:

W is the ideal work [Watts]
Q is the volumetric flow rate [m³/s]
ΔP is the pressure drop [Pa]

### For Maximum Velocity

$$\begin{matrix}W = ( 6.25 [ \frac{mL}{min} ] * \frac{1 * {10}^{- 6} [ {m}^{3} ]}{1 [ mL ]} * \frac{1 [ min ]}{60 [ s ]} ) * \\ 18814.1 [ Pa ] \\ = 0.00196 Watts\end{matrix} \qquad Eqn . 32$$

For Minimum Velocity

$$\begin{matrix}W = ( 3.13 [ \frac{mL}{min} ] * \frac{1 * {10}^{- 6} [ {m}^{3} ]}{1 [ mL ]} * \frac{1 [ min ]}{60 [ s ]} ) * \\ 19212.2 [ Pa ] \\ = 0.001 Watts\end{matrix} \qquad Eqn . 33$$

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration is by way of example, and the scope of the inventions is not limited to the exact details shown or described.

Although the foregoing detailed description has been described by reference to an exemplary embodiment, and the best mode contemplated for carrying out the present inventive concept has been shown and described, it will be understood that certain changes, modification or variations may be made in embodying the above invention, and in the construction thereof, other than those specifically set forth herein, may be achieved by those skilled in the art without departing from the spirit and scope of the invention, and that such changes, modification or variations are to be considered as being within the overall scope of the present invention. Therefore, it is contemplated to cover the present invention and any and all changes, modifications, variations, or equivalents that fall with in the true spirit and scope of the underlying principles disclosed and claimed herein. Consequently, the scope of the present invention is intended to be limited only by the attached claims, all matter contained in the above description and shown in the accompanying drawings shall be interpreted as illustrative and not in a limiting sense.

Having now described the features, discoveries and principles of the invention, the manner in which the invention is constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

What is claimed is:

1. A method of using a bicorporal partially subcutaneous positive displacement pump, said method comprising:
positioning an external component to within operable proximity of an internal component sufficient that a driving force can transfer from the external component to the internal component wirelessly, without physical connection, and without breaking a physical barrier located between the external component and the internal component;
wherein the internal component is comprised of a biocompatible material and located within a subject's body and the external component is located external to the subject's body and the physical barrier located between the external component and the internal component is at least one dermal layer of the subject's body; and
wherein the internal component comprises a fluid flow chamber in fluid communication with a fluid inlet and a fluid outlet, said fluid flow chamber including a positive displacement pump configured to use the driving force to displace a predetermined volume of fluid in through said inlet and out through said outlet in one or more cycles;
transferring the driving force from the external component to the internal component; and
selectively controlling the transfer of the driving force from the external component to the internal component to control the flow of predetermined volume of fluid from the inlet to the outlet;
wherein when the driving force is not transferred to the internal component, no fluid flows from the inlet to the outlet.

2. The method according to claim 1, wherein the step of selectively controlling the transfer of the driving force includes transferring the driving force for a predetermined period of time.

3. The method according to claim 1, wherein the step of selectively controlling the transfer of the driving force includes transferring the driving force for a predetermined number of positive displacement pump cycles.

4. The method according to claim 1, further comprising:
selectively controlling the transfer of the driving force from the external component to the internal component to control a flow of a second predetermined volume of fluid from the outlet to the inlet.

5. The method according to claim 1, wherein the positive displacement pump is one of a peristaltic pump, a piston pump, or a diaphragm pump.

6. The method according to claim 1, further comprising:
taking a predetermined volume of fluid in to the inlet of the internal component via a catheter from a remote location within the subject's body; and
discharging a predetermined volume of fluid from the outlet of the internal component via a catheter to a remote location within the subject's body.

7. The method according to claim 1, wherein the step of transferring the driving force is accomplished via magnetic induction.

8. The method according to claim 1, wherein the step of selectively controlling the transfer of the driving force is independent of fluid pressure.

9. A bicorporal partially subcutaneous positive displacement pump apparatus comprising:
an external component located external to a subject's body; and
an internal component comprised of a biocompatible material and located within the subject's body;
wherein a physical barrier disposed between the internal component and the external component, said physical barrier comprising at least one dermal layer of the subject's body;
wherein when the external component is positioned within a predetermined proximity of the internal component a driving force can transfer from the external component to the internal component wirelessly, without physical connection, and without breaking the physical barrier located between the external component and the internal component;
wherein the internal component comprises a fluid flow chamber in fluid communication with a fluid inlet and a fluid outlet, said fluid flow chamber comprising a positive displacement pump configured to use the driving force to displace a predetermined volume of fluid in through said inlet and out through said outlet in one or more cycles;
wherein said external component comprises a controller to selectively control the transfer of the driving force from the external component to the internal component to control the flow of predetermined volume of fluid from the inlet to the outlet; and
wherein when the driving force is not transferred to the internal component, no fluid flows from the inlet to the outlet.

10. The apparatus according to claim 9, wherein the controller controls the transfer of the driving force for a predetermined period of time.

11. The apparatus according to claim 9, wherein the controller controls the transfer of the driving force for a predetermined number of cycles.

12. The apparatus according to claim 9, wherein the controller controls the transfer of the driving force from the external component to the internal component to control a flow of a predetermined volume of fluid from the outlet to the inlet.

13. The apparatus according to claim 9, wherein the positive displacement pump is one of a peristaltic pump, a piston pump, or a diaphragm pump.

14. The apparatus according to claim 9, further comprising:
a catheter configured to take in a predetermined volume of fluid from a remote location within the subject's body through the inlet of the internal component; and
a catheter configured to discharge a predetermined volume of fluid from the outlet of the internal component to a remote location within the subject's body.

15. The apparatus according to claim 9, wherein the driving force is transferred from the external component to the internal component via magnetic induction.

16. The apparatus according to claim 9, wherein the controller controls the predetermined volume of fluid flow independent of fluid pressure.
